(19) United States
(12) Patent Application Publication
(10) Pub. No.: US 2026/0226014 A1
(43) Pub. Date: Aug. 6, 2026

(54) COMPOSITION COMPRISING 1,3,5-TRIAZINE, 2,4,6-TRIS[1,1'-BIPHENYL]-4-YL WITH LOWER AMOUNTS OF IMPURITIES

(71) Applicant: BASF SE, Ludwigshafen am Rhein (DE)

(72) Inventors: Thomas EHLIS, Muttenz (CH); Markus KNOBLOCH, Muttenz (CH); Helmut KRONEMAYER, Ludwigshafen am Rhein (DE); Ute HOPPE, Ludwigshafen am Rhein (DE); Johannes Felix HAUS, Ludwigshafen am Rhein (DE)

(21) Appl. No.: 19/151,461

(22) PCT Filed: Jan. 26, 2024
(86) PCT No.: PCT/EP2024/051902

§ 371 Date: Jul. 28, 2025

(30) Foreign Application Priority Data
Jan. 30, 2023 (EP) 23153984.2

(51) Int. Cl. C07D 251/24; A61K 8/49; A61Q 5/00; A61Q 17/04

(52) U.S. Cl. CPC C07D 251/24; A61K 8/4966; A61Q 5/00; A61Q 17/04; A61K 2800/49; C07B 2200/13

(57) ABSTRACT

A composition comprising 1,3,5-triazine, 2,4,6-tris[1,1′-biphenyl]-4-yl (TBPT) and a first impurity consisting of one or more halogen-comprising triazine and a second impurity consisting of biphenyl, wherein the amount of the first impurity is equal to or less than 900 ppm by weight with respect to the total weight of the composition, and wherein the amount of the second impurity is equal to or less than 4000 ppm by weight with respect to the total weight of the composition.

### TECHNICAL FIELD

The present invention relates to a composition comprising 1,3,5-triazine, 2,4,6-tris[1,1′-biphenyl]-4-yl with less impurities, in particular in a new crystalline form A, the process for its preparation as well as a cosmetic composition containing said composition comprising TBPT and the use of said composition comprising TBPT as UV filter.

### BACKGROUND

In order to comply with cosmetics regulations, purity standards are set for chemical ingredients of cosmetic products. Purity standards may have particular relevance for cosmetic products comprising chemical ingredients that may exhibit impurities, which may be unbeneficial for the health of the user. In particular, aromatic hydrocarbon impurities may cause side effects on the human or animal body, when present in a cosmetic product. To reduce undesired side effects from impurities, chemical ingredients of cosmetic should be produced in a manner that focuses on reducing said impurities.

A chemical ingredient that is widely used in cosmetic products is 1,3,5-triazine, 2,4,6-tris[1,1′-biphenyl]-4-yl (TBPT). Commonly, TBPT is used as a highly efficient, photostable filter against UVB and UVA II radiation in cosmetic products for protecting hair or skin of a human or animal from the damaging effects of UV radiation, such as anti-aging face care products or sun protection products. Besides, TBPT is known from electroluminescent devices (e.g. U.S. Pat. No. 6,225,467 B1, WO 2020/226300, WO 2020/231197).

In US 2004/0191191 A1, a synthetic procedure is described to obtain TBPT from cyanuric chloride and 1,1′-biphenyl in an aluminum chloride catalyzed reaction in 1,2-dichlorobenzene. Without adequate purification steps, there is a risk that residual starting material may remain in the final product and thus end up in a cosmetic product.

In order to avoid impurities that may occur during TBPT synthesis in e.g. cosmetic products, there is a need of reducing those already during synthesis or purification steps.

Hence, there is an ongoing need for a fast and efficient production process of TBPT. In this connection, it has been an object of the present invention to provide an optimized process for synthesizing, isolating and purifying TBPT.

### SUMMARY OF THE INVENTION

It is one object of the present invention to provide a composition with increased amounts of TBPT in a crystalline form which allows use as a UV filter. In particular, it is one object of the present invention to provide a composition having a high content of TBPT in crystalline form A.

It is another object of the present invention to provide a process for synthesizing, isolating and purifying of a crystalline form of TBPT. Furthermore, it is another object of the present invention to provide a process for providing TBPT having high amounts of crystalline form A.

In a first aspect, the present invention provides a composition comprising 1,3,5-triazine, 2,4,6-tris[1,1′-biphenyl]-4-yl (TBPT) and a first impurity consisting of one or more halogen-comprising triazine and a second impurity consisting of biphenyl, wherein the amount of the first impurity is equal to or less than 900 ppm by weight with respect to the total weight of the composition, and wherein the amount of the second impurity is equal to or less than 4000 ppm by weight with respect to the total weight of the composition.

In a second aspect, the present invention provides a crystalline form A of TBPT, which, in an X-ray powder diffractogram at room temperature (20° C.) using Cu-Kα radiation, shows at least 3 of the 5 following reflexes, given in 2θ values: 11.6±0.2, 17.8±0.2, 21.1±0.2, 23.4±0.2 and 24.4±0.2°θ.

In a third aspect, the present invention provides a crystalline form B of TBPT, which, in an X-ray powder diffractogram at room temperature using Cu-Kα radiation, shows at least 3 of the 5 following reflexes, given in 2θ values: 8.6±0.2, 10.9±0.2, 17.5±0.2, 18.5±0.2, 19.5±0.2, 19.9±0.2, and 20.7±0.2°θ.

In a fourth aspect, the present invention provides a process for interconverting crystalline form B of 2,4,6-tris(biphenyl-4-yl)-1,3,5-triazine (TBPT) into crystalline form A of TBPT comprising the steps of a) providing a composition C_{B} comprising TBPT in crystalline form B; b) mixing the composition C_{B} in a solvent (S1) to obtain a first mixture; c) stirring the first mixture at a temperature T₁ for a time t₁ and thereby obtaining TBPT in crystalline form A, and thereby providing a second mixture; d) isolating a composition C_{A} comprising TBPT in crystalline form A from the second mixture.

In a fifth aspect, the present invention provides a cosmetic product comprising the composition according to the first aspect of the invention, preferably wherein more than 80 wt-%, preferably 100 wt-% of the TBPT is present in crystalline form A in a micronized form.

In a sixth aspect, the present invention provides a use of the composition according to the first aspect of the present invention as a UV filter in a cosmetic product for protecting hair and/or skin of a subject from the damaging effects of UV radiation, wherein preferably more than 80 wt-%, preferably 100 wt-% of the TBPT is present in crystalline form A.

The present invention will be described with respect to particular embodiments and with reference to certain examples, but the invention is not limited thereto, and it is only defined by the appending claims.

### BRIEF DESCRIPTION OF THE DRAWINGS

FIG. 1 shows a DSC diagram of RE1. TBPT is indicated by the endothermic peak at 221-256° C. with ΔH=39-43 J/g.

FIG. 2 shows a PXRD pattern of RE1, Cu Kα radiation.

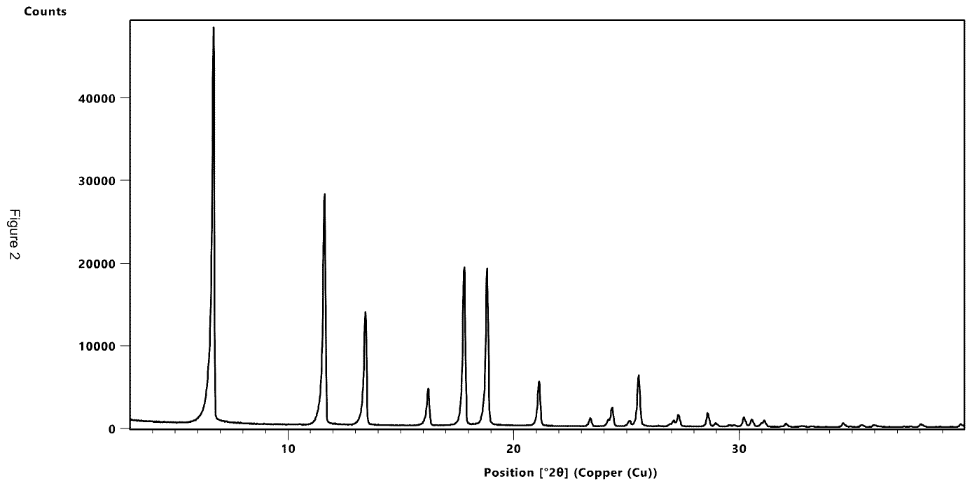

FIG. 3 shows a PXRD pattern of IE1, Cu Kα radiation.

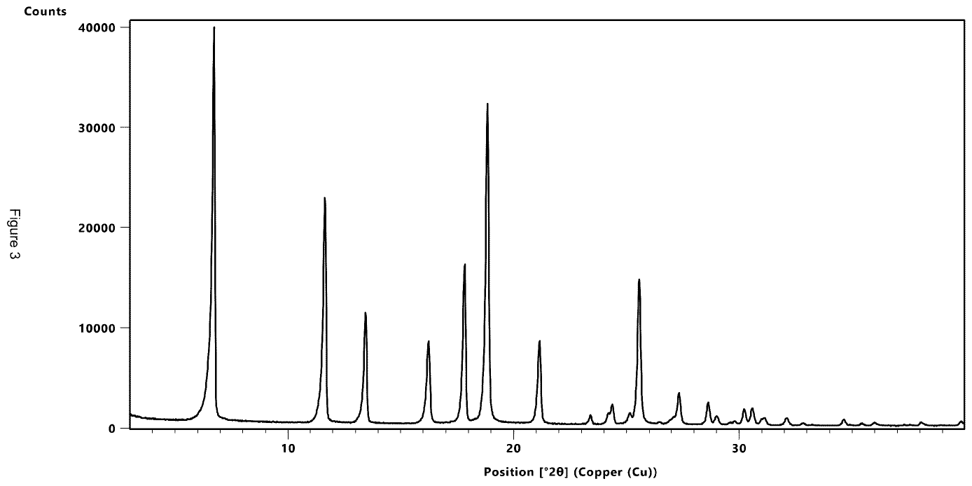

FIG. 4 shows a PXRD pattern of CE3, Cu Kα radiation.

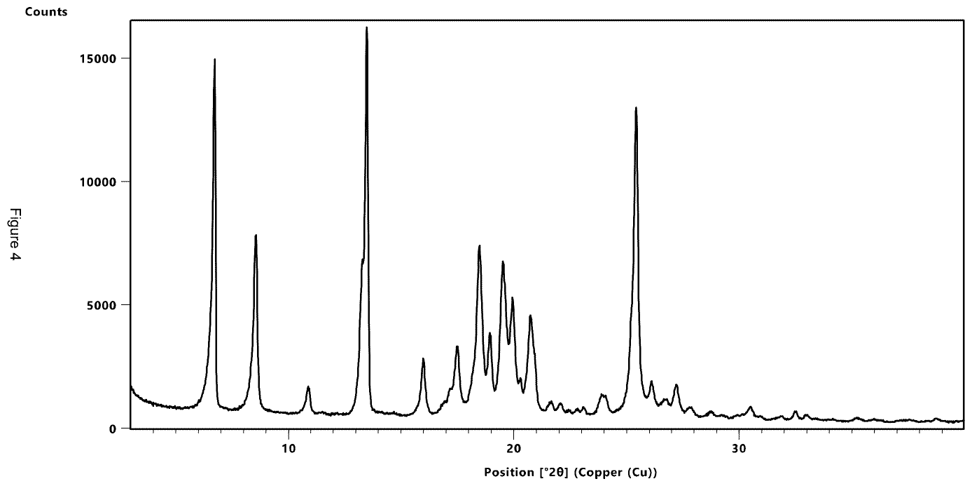

FIG. 5 shows a PXRD pattern of CE2, Cu Kα radiation.

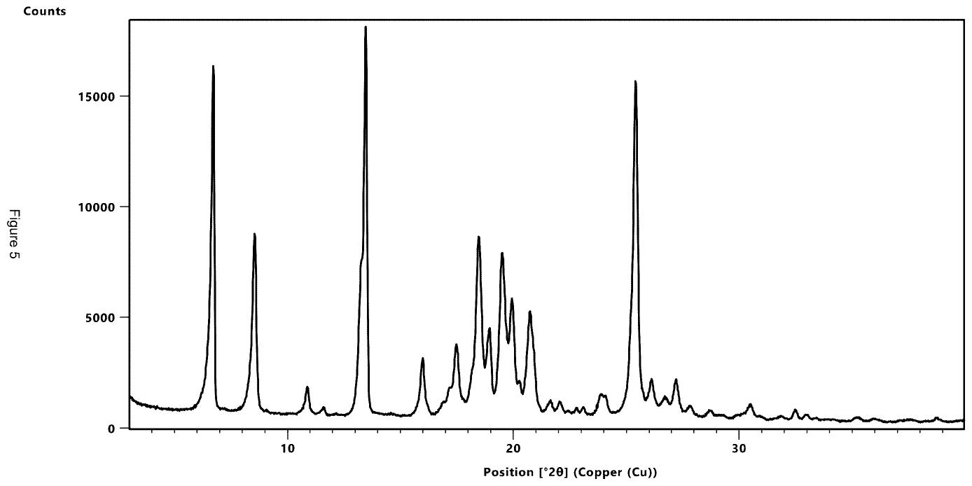

FIG. 6 shows a PXRD pattern comparison of forms A (top) and B (bottom), Cu Kα radiation.

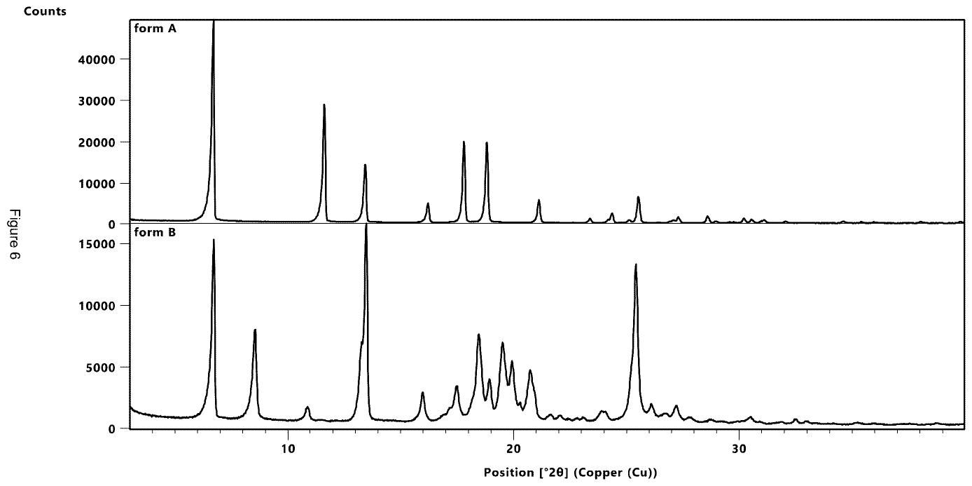

FIG. 7 shows a comparison of the UV absorption spectra of TBPT forms A and B.

### DEFINITIONS

Before describing in detail exemplary embodiments of the present invention, definitions important for understanding the present invention are given.

Terms as set forth hereinafter are generally to be understood in their common sense unless indicated otherwise.

The term “comprising” does not exclude other elements. For the purposes of the present invention, the term “consisting of” is considered to be a preferred embodiment of the term “comprising”. If hereinafter a group is defined to comprise at least a certain number of embodiments, this is also to be understood to disclose a group, which preferably consists only of these embodiments.

Where an indefinite or definite article is used when referring to a singular noun, e.g. “a”, “an” or “the”, this includes a plural of that noun unless specifically stated otherwise.

The term “at least one” means numerically “one or more”. In a preferred embodiment, the term numerically means “one”.

The terms “include” and “comprising” mean that there may be other components in addition to those mentioned. These terms are meant inclusively and therefore include “consisting of”. “Consisting of” is meant conclusively and means that no further constituents may be present. In an embodiment, the terms “comprise” or “comprising” mean “consisting of”.

Terms like “obtainable” and “obtained” are used interchangeably. This, e.g., means that, unless the context clearly dictates otherwise, the term “obtained” does not mean to indicate that e.g. an embodiment must be obtained by e.g. the sequence of steps following the term “obtained” even though such a limited understanding is always included by the terms “obtained” as a preferred embodiment.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only and is not intended to limit the scope of the present invention that will be limited only by the appended claims.

The terms “about” or “approximately” allow a deviation from the indicated numerical value of ±20%, preferably ±15%, more preferably ±10%, even more preferably ±5%, in a strongly preferred embodiment ±1%, and in the most preferred embodiments “about” and “approximately” mean “exactly”.

A range delimited by numbers, e.g., “from 80° C. to 120° C.” means that the two corner values and each value within that range are individually disclosed.

The term “room temperature”, as used herein, relates to 20° C. The term “standard pressure”, as used herein, relates to 1013 mbar.

The organic moieties mentioned in the above definitions of the variables are—like the term halogen—collective terms for individual listings of the individual group members. The prefix Cn-Cm indicates in each case the possible number of carbon atoms in the group.

The term “halogen” denotes in each case fluorine, bromine, chlorine, or iodine, in particular fluorine, chlorine, or bromine.

The term “crystalline form” in relation to TBPT according to the present disclosure comprises in particular the crystalline form A and crystalline form B. However, it is not excluded that there may be additional crystalline forms.

The term “polymorphism” describes an enantiotropic relationship that implies that each form has a range of temperature over which it is stable with respect to the other and a transition point at which the forms are equistable and in principle interconvertible. Above that temperature, the thermodynamic tendency is to the formation exclusively of the form stable at the higher temperature. Below the transition temperature, the low-temperature form is the only stable one with respect to the other, although there is usually a greater tendency for the high temperature form to become frozen-in than for a low-temperature form to persist beyond its stability range. Forms outside their range of stability are described here as metastable.”

The term “alkyl” as used herein denotes in each case a straight-chain or branched alkyl group having usually from 1 to 5 carbon atoms, preferably from 1 to 4 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, 1,1-dimethylpropyl, and 1,2-dimethylpropyl. Methyl, ethyl, n-propyl, iso-propyl, and iso-butyl, are particularly preferred.

The term “aryl” preferably includes 6-membered aromatic carbocyclic rings based on carbon atoms as ring members. A preferred example is phenyl. In aromatic ring systems, the Hückel (4n+2) rule is fulfilled.

The term “purity” in this context relates to the purity of a batch that was obtained by a “production process”, wherein said a product obtained from a production process typically comprises impurities. The purity of a batch may be determined by quantifying the TBPT content in a batch by HPLC. The experimental details for the method for quantification is further described in the examples given below. According to the present disclosure, TBPT may show a purity of e. g. 95 wt-%, 98 wt-%, 98.5 wt-%, 99.0 wt-% or even 100 wt-%. As such, a purity of 98.5 wt-% may be represent a mixture of 98.5 wt-% pure TBPT and 1.5 wt-% of impurities.

The term “impurities” as used herein relates to chemical compounds in a batch TBPT obtained by a production process that are not TBPT. As such, the term “impurities” relates to inorganic and organic impurities. Particularly relevant impurities in the context of the present disclosure are impurities from remaining starting material, intermediate products or solvents that may be used or occur during a production process. Among those impurities there may be problematic impurities due to toxicological concerns, which are for example aromatic hydrocarbons, such as biphenyl or xylene, or halogenated triazines, such as 2,4-bis([1,1′-biphenyl]-4-yl)-6-chloro-1,3,5-triazine (BBCT). The list of “impurities” cannot be understood as a comprehensive or conclusive list of impurities. In the context of this disclosure, certain known “impurities” are quantified by GC or HPLC. The experimental details for the method for quantification is further described in the examples given below.

The term “xylene” as referred herein covers all xylene isomers and a mixture there of comprising 1,2-dimethylbenzene, 1,3-dimethylbenzene and 1,4-dimethylbenzene, corresponding to ortho-xylenes, meta-xylenes, para-xylenes, also o-xylenes, m-xylenes, and p-xylenes.

The term “production process” refers to a process that may comprise the steps of synthesizing, isolating and purifying. The production process may only consist of one or two of these steps and optionally comprise additional steps. The production process is thus not to be understood to be limited to these steps or even the order of the steps, if not stated otherwise.

The term ‘TBPT’ as used herein has to be understood as abbreviation for the chemical compound name 1,3,5-triazine, 2,4,6-tris[1,1′-biphenyl]-4-yl (CAS Number: 31274-51-8, also: 2,4,6-tris([1,1′-biphenyl]-4-yl)-1,3,5-triazine), which is described according to formula (I).

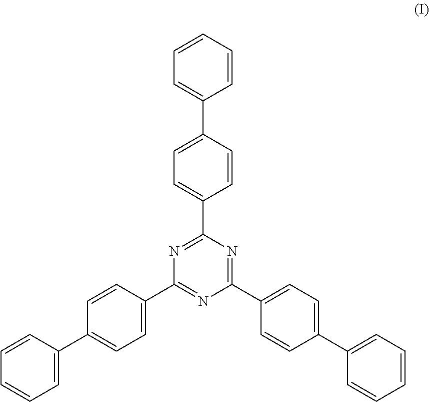 [SMILES: c1ccc(-c2ccc(-c3nc(-c4ccc(-c5ccccc5)cc4)nc(-c4ccc(-c5ccccc5)cc4)n3)cc2)cc1]

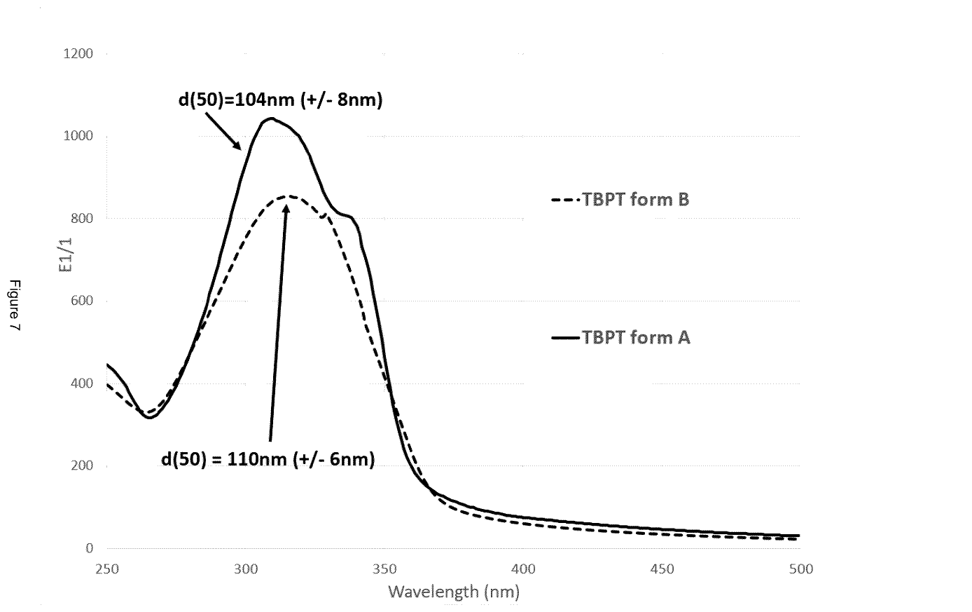

TBPT may occur in amorphous form or crystalline form and mixtures thereof. Compound of formula (I) can exist in at least two crystal modifications or in mixtures of the two of more crystalline states. Polymorph type A can be distinguished from polymorph type B by powder X-ray powder diffraction (XRD) and differential scanning calorimetry (DSC). Only polymorph type A is suitable for sunscreen application due to the specific UV absorption properties (cf. FIG. 7). Moreover, polymorph type B is metastable at room temperature. Methods and data are provided for polymorphs type A and B.

The term “raw TBPT” or “TBPT raw” according to the present disclosure, refers to TBPT as a crude product. “Raw TBPT” or “TBPT raw” may be obtained from a production process resulting in a product with a higher level of impurities. TBPT raw may comprise TBPT in the crystalline form A.

### DETAILED DESCRIPTION OF THE INVENTION

Hence, in a first aspect the present invention relates to a composition comprising TBPT and a first impurity consisting of one or more halogen-comprising triazine and a second impurity consisting of biphenyl, wherein the amount of the first impurity is equal to or less than 900 ppm by weight with respect to the total weight of the composition, and wherein the amount of the second impurity is equal to or less than 4000 ppm by weight with respect to the total weight of the composition.

Both the first and the second impurity, but in particular the second impurity, i.e. biphenyl, are side products of the usually used production process of TBPT as described i.e. in US 2004/0191191 A1. Both impurities comprise aromatic entities, which are known to be capable of increasing the risk of diseases such as cancer. Hence, the first aspect of the present invention provides a composition comprising TBPT with lower amounts of such impurities than realized in the prior art.

Hence, preferably, the halogen-comprising triazine of the first impurity of the composition of the first aspect of the present invention is selected from the group consisting of chlorine-comprising triazines, bromine-comprising triazines, and a mixture thereof. The halogen-comprising triazines, such as the chlorine-comprising triazine and bromine-comprising triazine include 1,3,5-triazines, in particular phenyl substituted, such as 2,4,6-trisphenyl, or biphenyl substituted, such as 2,4-di([1,1′-biphenyl]-4-yl), 1,3,5-triazines in which one or more hydrogen atoms are replaced by halogen atoms, such as chloro or bromo, for example on the 1,3,5-triazine ring or the phenyl rings. In various embodiments, the first impurity more preferably is 1,3,5-triazine, 2,4,6-tris(4-bromophenyl) and/or 1,3,5-triazine, 2,4-bis([1,1′-biphenyl]-4-yl)-6-chloro (BBCT), and most preferably is 1,3,5-triazine, 2,4-bis([1,1′-biphenyl]-4-yl)-6-chloro (BBCT).

Especially preferably, the amount of the first impurity in the composition of the first aspect of the present invention is equal to or less than 800 ppm by weight with respect to the total weight of the composition, more preferably equal to or less than 600 ppm by weight, and most preferably equal to or less than 400 ppm by weight. This further reduces the risk of diseases if the composition is used, i.e., in cosmetic products.

In a preferred embodiment of the first aspect of the present invention the amount of the first impurity in the composition of the present invention is equal to or more than 0.01 ppm by weight with respect to the total weight of the composition, preferably is equal to or more than 0.1 ppm by weight with respect to the total weight of the composition, more preferably is equal to or more than 1 ppm by weight, still more preferably is equal to or more than 10 ppm by weight and most preferably is equal to or more than 100 ppm by weight.

It is understood that the amount of the first impurity given above can refer to the total amount of first impurities, i.e. all compounds that are halogen-containing triazines in the sense of the present invention, or to the more specifically defined first impurities described herein. In various embodiments, the amount may thus also refer to one of the more specifically defined first impurities defined above, such as 1,3,5-triazine, 2,4,6-tris(4-bromophenyl) and/or 1,3,5-triazine, 2,4-bis([1,1′-biphenyl]-4-yl)-6-chloro (BBCT). It may however be preferred that the given maximum amount for the first impurity covers the total amount of all compounds that qualify as halogen-comprising triazines. These amounts may also be combined such that the total amount of halogen-comprising triazines is equal to or less than 900 ppm by weight with respect to the total weight of the composition and the amount of a specific impurity, such as 1,3,5-triazine, 2,4-bis([1,1′-biphenyl]-4-yl)-6-chloro (BBCT), is equal to or less than 400 ppm by weight.

For the same reasons as set out above for the first impurity, preferably, the amount of the second impurity in the composition of the first aspect of the invention is equal to or less than 3000 ppm by weight with respect to the total weight of the composition, more preferably equal to or less than 600 ppm by weight, and most preferably equal to or less than 300 ppm by weight.

In a further preferred embodiment of the first aspect of the invention, the amount of the second impurity in the composition is equal to or more than 0.01 ppm by weight with respect to the total weight of the composition, more preferably is equal to or more than 0.1 ppm by weight, still more preferably is equal to or more than 1 ppm by weight, and even still more preferably is equal to or more than 10 ppm by weight, and most preferably is equal to or more than 100 ppm by weight.

In the production process for TBPT as described in US 2004/0191191 A1, usually an aromatic solvent is used. This aromatic solvent can be a condensed aromatic system. Examples for such solvents are benzene, toluene, xylene, ethylbenzene, naphthalene, fluorene, and mixtures thereof. However, also such aromatic compounds can be problematic in view of diseases caused by application of a composition comprising TBPT in a cosmetic product. Thus, it is also a desire of the present invention to further reduce the amount of such solvent in the composition comprising TBPT.

Thus, preferably, the composition according to the first aspect of the present invention further comprises a third impurity, the third impurity comprising, preferably consisting of, an aromatic hydrocarbon, wherein the third impurity does not comprise biphenyl. This means that the third impurity may comprise an aromatic hydrocarbon or consist of an aromatic hydrocarbon. Preferably, the one or more aromatic hydrocarbon is selected from the group consisting of monocyclic-aromatic hydrocarbons, polycyclic aromatic hydrocarbons (PAH), and mixtures thereof, preferably is selected from the group consisting of alkyl-substituted benzene compounds (Cn-benzenes), condensed aromatic hydrocarbons, and mixtures thereof, more preferably is selected from the group consisting of benzene, toluene, xylene, ethylbenzene, naphthalene, fluorene, and mixtures thereof, and most preferably is xylene.

In a preferred embodiment of the first aspect of the present invention, the amount of the third impurity in the composition is equal to or lower than 4000 ppm by weight with respect to the total weight of the composition, preferably equal to or lower than 800 ppm by weight, and more preferably equal to or lower than 400 ppm by weight. Preferably, the amount of the third impurity in the composition of the present invention is equal to or more than 5 ppm by weight with respect to the total weight of the composition, preferably equal to or more than 10 ppm by weight, and more preferably equal to or more than 20 ppm by weight.

Similar to the definition of the amounts of the first impurity given above, it is understood that the amount of the third impurity can refer to the total amount of compounds that qualify as third impurities, i.e. all compounds that are aromatic hydrocarbons in the sense of the present invention, or to the more specifically defined third impurities described herein. In various embodiments, the amount may thus also refer to one of the more specifically defined third impurities defined above, such as xylene. It may however be preferred that the given maximum amount for the third impurity covers the total amount of all compounds that qualify as aromatic hydrocarbons. These amounts may also be combined such that the total amount of aromatic hydrocarbons is equal to or less than 4000 ppm by weight with respect to the total weight of the composition and the amount of a specific impurity, such as xylene, is equal to or less than 400 ppm by weight.

Preferably, the composition according to the first aspect of the present invention comprises the TBPT in an amount in the range of from 98.0 wt-% to 99.9 wt-% with respect to the total weight of the composition, more preferably in the range of from 98.5 wt-% to 99.9 wt-%.

Due to the process of the third aspect of the present invention detailed further below, the composition of the first aspect of the invention may comprise a further compound, wherein the further compound may be a solvent (S1) having a boiling point B₁ at a pressure p_{B1} of 1 bar at a temperature T_{B1} equal to or lower than 235° C., preferably lower than 230° C., more preferably equal to or lower than 200° C., equal to or lower than 180° C., equal to or lower than 160° C., most preferably equal to or lower than 140° C. Furthermore, preferably, the further compound has a melting point M₂ at a pressure p_{B2} of 1 bar at a temperature T_{B2} equal to or lower than 20° C.

Preferably, the further compound is a solvent (S1), preferably polar solvent, more preferably a polar organic compound or water. Preferably, the polar organic compound is selected from the group consisting of esters, alcohols, ethers, aldehydes, ketones, and mixtures thereof. Especially preferably, the polar organic compound is a methyl ketone or an alcohol, most preferably acetone, methyl ethyl ketone, or ethanol. Hence, preferably, the further compound is selected from the list consisting of acetone, methyl ethyl ketone, water, ethanol, or mixtures thereof.

Further, it has surprisingly been found that TBPT occurs in two crystalline polymorph forms, herein referred to as polymorph types A and B or (crystalline) forms A and B. Crystalline form A can be distinguished from crystalline form B by X-ray powder diffraction spectroscopy. Thereby, it was observed that the ratio of the amounts of these two crystalline forms varies depending on the conditions used in the production process of TBPT. Besides, it was observed that in particular crystalline form A shows thermal stability at temperatures around 15° C. to 45° C. Hence, while crystalline form B shows also long storage times, crystalline form A of TBPT has improved storability in comparison to form B. Furthermore, form A of TBPT is suitable for sunscreen application due to the specific UV filter properties, i.e., the UV absorption spectrum of form A (cf. FIG. 7).

Thus, in a second aspect of the present invention, the composition comprises preferably at least a part of the TBPT, preferably the total TBPT, in a crystalline form A, which, in an X-ray powder diffractogram at room temperature using Cu-Kα radiation, shows at least 3 of the 5 following reflexes, given in 2θ values: 11.6±0.2, 17.8±0.2, 21.1±0.2, 23.4±0.2 and 24.4±0.2°θ. More preferably, the crystalline form A shows in an X-ray powder diffractogram at room temperature using Cu-Kα radiation, at least the following three reflexes 11.6±0.2, 17.8±0.2 and 21.1±0.2°θ, and preferably shows at least the following five reflexes 11.6±0.2, 17.8±0.2, 21.1±0.2, 23.4±0.2 and 24.4±0.2°θ.

It has been found out that in comparison to the form B as also found as a metastable polymorph of TBPT, form A provides the optimal UV absorption spectrum for sunscreen applications (cf. FIG. 7). Hence, it is beneficial to have most of the TBPT present in the composition in form A. Thus, preferably, the amount of TBPT in crystalline form A in the composition is more than 50 wt-% with respect to the total weight of the TBPT in the composition, preferably more than 60 wt-%, more than 65 wt %, more than 70 wt-%, more than 75 wt-%, more than 80 wt-%, more than 81 wt-%, more than 82 wt-%, more than 83 wt-%, more than 84 wt-%, more than 85 wt-%, more than 86 wt-%, more than 87 wt-%, more than 88 wt-%, more than 89 wt-%, more than 90 wt-%, more than 91 wt-%, more than 92 wt-%, more than 93 wt-%, more than 94 wt-%, more than 95 wt-%, more than 96 wt-%, more than 97 wt-%, more than 98 wt-%, more than 99 wt-%, and most preferably 100 wt-%.

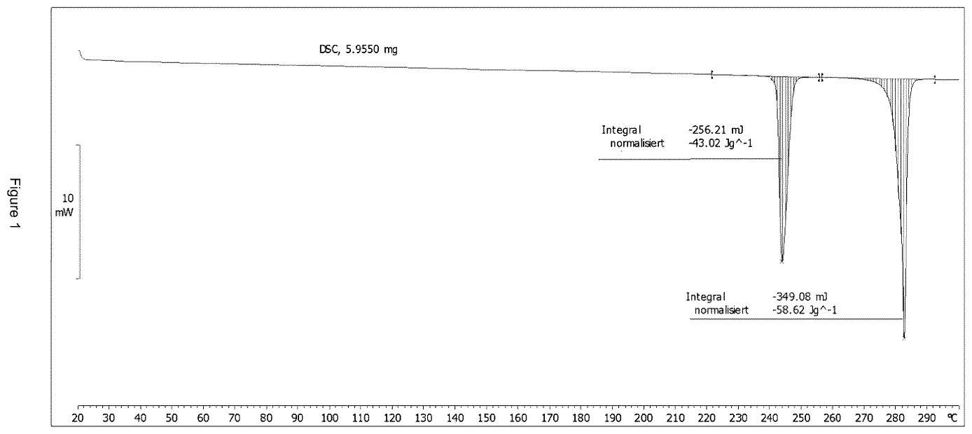

Preferably, the crystalline form A exhibits an endothermic peak in a DSC curve from 221° C. to 256° C., wherein the DSC curve is measured by a differential scanning calorimeter at a scan rate of 10° C. per minute (cf. FIG. 1). More preferably, the crystalline form A, which in a DSC curve exhibits an endothermic peak in a DSC curve from about 221° C. to about 256° C., further preferably wherein the enthalpy ΔH measured at said endothermic peak is in the range from about 35 J/g to about 50 J/g, more preferably wherein the enthalpy ΔH measured at said endothermic peak is in the range from about 38 J/g to about 45 J/g.

It has been further found out that the polymorphic forms A and B are enantiotropic to each other. The transition temperature between the two forms is in the range 230-250° C. Heating TBPT to this high temperature induces a phase transformation from the low temperature stable form A to the high-temperature stable form B. The two forms presumably represent an enantiotropic system of polymorphic forms, but the transformation is not readily reversed upon cooling.

Moreover, in a third aspect of the present invention, the composition comprises preferably at least a part of the TBPT, preferably the total TBPT, is comprised in the composition in a crystalline form B, which, in an X-ray powder diffractogram at room temperature using Cu-Kα radiation, shows at least 3 of the 5 following reflexes, given in 2θ values: 8.6±0.2, 10.9±0.2, 17.5±0.2, 18.5±0.2, 19.5±0.2, 19.9±0.2, and 20.7±0.2°θ.

Preferably, the crystalline form B exhibits an endothermic peak in a DSC curve from 282° C. to 293° C., wherein the DSC curve is measured by a differential scanning calorimeter at a scan rate of 10° C. per minute. Preferably, in some embodiments, the TBPT is comprised in crystalline form B in the composition a weight ratio of more than 50 wt-% with respect to the total weight of the TBPT in the composition, preferably more than 60 wt-%, more than 65 wt %, more than 70 wt-%, more than 75 wt-%, more than 80 wt-%, more than 81 wt-%, more than 82 wt-%, more than 83 wt-%, more than 84 wt-%, more than 85 wt-%, more than 86 wt-%, more than 87 wt-%, more than 88 wt-%, more than 89 wt-%, more than 90 wt-%, more than 91 wt-%, more than 92 wt-%, more than 93 wt-%, more than 94 wt-%, more than 95 wt-%, more than 96 wt-%, more than 97 wt-%, more than 98 wt-%, more than 99 wt-%, and most preferably 100 wt-%.

Form A is suitable for sunscreen application due to the specific UV absorption properties. However, biphenyl and xylene in TBPT raw are problematic aromatic hydrocarbons due to toxicological concerns. It is not possible to significantly reduce the impurities by multiple recrystallizations from xylene or from xylene/biphenyl mixtures because the low-temperature stable form A readily incorporates small amounts of almost any solvent. However, further purification of TBPT raw is mandatory at least for therapeutic applications or personal care applications (e.g., sunscreen applications). Hence, there is the need for finding a process of not only purifying raw TBPT compositions, but also interconverting TBPT present in form B to form A in such compositions.

Thus, in a fourth aspect, the present disclosure provides a process for interconverting crystalline form B of 2,4,6-tris(biphenyl-4-yl)-1,3,5-triazine (TBPT) into crystalline form A of TBPT comprising the steps of a) providing (in a TBPT providing step) a composition C_{B} comprising TBPT in crystalline form B; b) mixing (in a first mixing step) the composition C_{B} in a solvent (S1) to obtain a first mixture; c) stirring (in a stirring step) the first mixture at a temperature T₁ for a time t₁ and thereby obtaining TBPT in crystalline form A, and thereby providing a second mixture; and d) isolating (in an isolating step) a composition C_{A} comprising TBPT in crystalline form A from the second mixture.

Without being bound by theory it is believed the that the conversion of form B to form A of the present invention is accelerated by a process called “solvent-interactive transformation”.

In general, the process of the fourth aspect of the present invention can be used to purify and interconvert any raw TBPT. However, it is preferably carried out with a composition comprising TBPT prepared with a process involving biphenyl and/or xylene. Hence, in the process according to the fourth aspect of the present invention, the TBPT providing step preferably further comprises the step of reacting a 2,4,6-trihalogen-1,3,5-triazine with biphenyl. More preferably, the step of reacting a 2,4,6-trihalogen-1,3,5-triazine with biphenyl comprises the steps of a′) reacting cyanuric chloride with biphenyl in heptane upon the addition of hydrochloric acid and aluminum chloride at temperatures in the range from about 90° C. to about 120° C.; b′) removing heptane from the reaction mixture under reduced pressure; c′) adding water, sodium hydroxide and xylene to the reaction mixture.

Preferably, the step of reacting a 2,4,6-trihalogen-1,3,5-triazine with biphenyl further comprises one or more of the following optional steps:
d′) optionally separating the organic phase from the aqueous phase, and washing the organic phase with water, separating organic phase from the aqueous phase and removing water completely from the organic phase by azeotropic distillation, heating the suspension at a temperature from about 130° C. to about 160° C.,
e′) optionally filtering the suspension at a temperature from about 130° C. to about 160° C., cooling the filtrate to a temperature from about 40° C. to about 60° C. and allow TBPT to crystallize, isolate the product by filtration, wash the filtered product with a solvent (S2) and dry the product to obtain raw TBPT.

Preferably, in the step of reacting a 2,4,6-trihalogen-1,3,5-triazine with biphenyl the 2,4,6-trihalogen-1,3,5-triazine is cyanuric chloride. Further preferably, in the step of reacting a 2,4,6-trihalogen-1,3,5-triazine with biphenyl a solvent is used in the reaction mixture. In another preferred embodiment, the solvent is a non-polar solvent. In particular, linear, branched, or cyclic aliphatic hydrocarbons or aromatic hydrocarbons comprising C₁-C₂₀ carbon atoms optionally substituted with, e.g., an C₁-C₃ alkyl group, alkyl-aryl ethers, alkyl-alkyl ethers may be used optionally substituted with an alkoxy, halide or carbonyl moiety. In a preferred embodiment, a linear aliphatic hydrocarbon is used, most preferably hexane is used. The step of reacting a 2,4,6-trihalogen-1,3,5-triazine with biphenyl can be carried out in absence of a solvent like 1,2-dichlorobenzene.

In one embodiment, in the step of reacting a 2,4,6-trihalogen-1,3,5-triazine with biphenyl a catalyst is used. Catalysts may act as Lewis acid. Catalysts may be selected from the group consisting of acidic halides, metal alkyl and alkoxides, proton acids, acidic oxides, cation-exchange resins and mixtures thereof, preferably selected from the group consisting of AlCl₃, AlBr₃, BF₃, BCl₃, BBr₃, BeCl₂, CdCl₂, ZnCl₂, GaCl₃, GaBr₃, FeCl₃, SbCl₃, BiCl₃, TiCl₄, ZrCl₄, SnCl₄, UCl₄, SbCl₅, and mixtures thereof. In a preferred embodiment, the catalyst is used in stoichiometric amounts or in excess. As co-catalysts alcohols, water, HCl, HF, H₂SO₄, H₂PO₄, RCOOH (organic acids), sulfonic acids like for example p-toluene sulfonic acid may be used. Most preferably, gaseous HCl is used as co-catalyst. The co-catalysts can also be used in stoichiometric amounts or in excess. The reaction of the step of reacting a 2,4,6-trihalogen-1,3,5-triazine with biphenyl runs particularly well when gaseous HCl is discharged into the reaction mixture. Preferably, in the step of reacting a 2,4,6-trihalogen-1,3,5-triazine with biphenyl, the reaction temperature is from about −10 to about 250° C., more preferably the reaction temperature is in the range from about 5 to about 150° C., most preferably from about 70 to about 130° C.

Furthermore, the step of reacting a 2,4,6-trihalogen-1,3,5-triazine with biphenyl can be carried out in ionic fluids like for example 1-butyl-pyridinium chloride-aluminum (HI) chloride and 1-butyl-3-methylimidazolium chloride-aluminum (HI) chloride (See, e.g, 1-ethyl-3-methylimidazolium halogenoaluminate ionic liquids as solvents for Friedel-Crafts acylation reactions of ferrocene. Journal of the Chemical Society, Dalton Transactions: Inorganic Chemistry 1999 (1), 63).

Preferably, in the process according to the fourth aspect of the present invention the TBPT providing step further comprises the step of treating the non-purified composition with a second solvent (S2) (cf. step e′)). Furthermore, also preferably, also the isolating step of the process of the fourth aspect of the present invention comprises the steps of filtering and washing the crystals with at least one second solvent (S2).

In a preferred embodiment of the fourth aspect of the present invention, the second solvent (S2) is not miscible with water, preferably is a hydrocarbon solvent, more preferably is selected from the list consisting of toluene, mesitylene, xylene, and mixtures thereof. These additional steps further improve the purity of the product in view of the first and the second impurity comprised in the non-purified composition.

Moreover, preferably, the process according to the fourth aspect of the present invention further comprises the steps of e) providing (in a second providing step) a non-purified composition comprising TBPT and at least a first impurity, the first impurity consisting of one or more halogen-comprising triazine, wherein the amount of the first impurity in the composition is equal to or higher than 950 ppm by weight with respect to the total weight of the composition; and f) heating (in a heating step) the non-purified composition to a transition temperature T_{T} in the range of from 235° C. to 280° C. This step assures that residual solvents and other molecules can be removed from the composition by evaporation. This further helps purifying the composition of the first aspect of the present invention by the process according to the fourth aspect of the present invention.

Preferably, in the stirring step the temperature T₁ is in the range of from 10° C. to 180° C., more preferably 50 to 140° C. Likewise, preferably, in the stirring step the pressure p, is in the range of from 900 mbar to 4000 mbar, preferably from 950 mbar to 3000 mbar, and most preferably from 970 mbar to 2700 mbar.

In a preferred embodiment of the fourth aspect of the present invention, the first mixture of the first mixing step comprises the solvent (S1) in an amount in the range of from 5 wt-% to 50 wt-% with respect to the total weight of the first mixture, preferably in the range of from 5 to 40 wt-%, 5 to 35 wt-%, 6 to 34 wt-%, 7 to 33 wt-%, 8 to 32 wt-%, 9 to 31 wt-%, 10 to 30 wt-%. Preferably, in the stirring step the time t₁ is in the range of from 1 h to 12 h, preferably 2 to 11 h, and most preferably 3 to 10 h. Preferably, the first mixing step is a mixing step involving intense mixing, most preferably the first mixing step comprises, preferably consists of, a kneading step. Hence, most preferably, the first mixing step is carried out in a kneader reactor.

Thus, most preferably, the process of the fourth aspect of the present invention is carried out in a kneader reactor.

In a preferred embodiment of the fourth aspect of the present invention, the isolation step comprises heating the first mixture to a temperature T₂ and/or reducing the pressure to a pressure p₂ and thereby removing solvent (S1) to obtain a composition C_{A}. Preferably, the temperature T₂ is in the range of from 100 to 150° C., more preferably 120 to 140° C. Likewise, the pressure p₂ is preferably in the range of from 10 to 300 mbar, more preferably 50 to 150 mbar.

Preferably, the solvent (S1) has a boiling point B₁ at a pressure p_{B1} of 1 bar at a temperature T_{B1} equal to or lower than 235° C., preferably lower than 230° C., more preferably equal to or lower than 200° C., equal to or lower than 180° C., equal to or lower than 160° C., most preferably equal to or lower than 140° C. Furthermore, preferably, the solvent (S1) has a melting point M₂ at a pressure p_{B2} of 1 bar at a temperature T_{B2} equal to or lower than 20° C. This ensures that during the process, no further crystalline form B of TBPT is formed.

Preferably, the solvent (S1) is a polar solvent, more preferably a polar organic compound or water. Preferably, the polar organic compound is selected from the group consisting of esters, alcohols, ethers, aldehydes, ketones, and mixtures thereof. Especially preferably, the polar organic compound is a methyl ketone or an alcohol, most preferably acetone, methyl ethyl ketone, or ethanol. Hence, preferably, the solvent (S1) is selected from the list consisting of acetone, methyl ethyl ketone, ethanol, water, or mixtures thereof. It has surprisingly been found that such solvents are suitable for the process of the present invention, although TBPT has minimal solubility in these solvents.

Solvent residues of halogenated hydrocarbons (US 2004/191191) can be removed by using the process of the fourth aspect of the present invention. In case that the synthesis of TBPT is performed without solvent (US 2004/191191), residues of biphenyl can be removed by using the process of the fourth aspect of the present invention. Also, in case that the synthesis of TBPT is performed without AlCl₃, residues of biphenyl can be removed by using the process of the fourth aspect of the present invention.

In a fifth aspect, the present invention provides a cosmetic product comprising the composition according to the first aspect of the invention, preferably wherein the TBPT is present in crystalline form A in a micronized form.

In a sixth aspect, the present invention provides a use of the composition according to the first aspect of the present invention as a UV filter in a cosmetic product for protecting hair and/or skin of a subject from the damaging effects of UV radiation.

### EXAMPLES

The present invention is further illustrated by the following examples.

### List of Abbreviations

DSC differential scanning calorimetry
PXRD powder X-ray diffraction spectroscopy
GC gas chromatography
HPLC high-performance liquid chromatography
FID flame ionization detector (FID)
DAD diode array detector
g gram(s)
min minute(s)
h hour(s)
T temperature
K Kelvin
° C. degrees Celsius
ΔH enthalpy change
J joule(s)
m meter
L liter
k kilo
m milli
μ micro
TBPT 1,3,5-triazine, 2,4,6-tris[1,1′-biphenyl]-4-yl
BBCT 2,4-bis([1,1′-biphenyl]-4-yl)-6-chloro-1,3,5-triazine
approx. approximately
PSD particle size distribution

### Methods

### a) Characterization of TBPT Polymorphs

DSC experiments were performed in open pans. In this case, volatile solvents and impurities evaporate irreversibly. Scanning rate 10 K/min, 20° C.-300° C., N₂ 50 ml/min. Instrument: DSC3+ Module, Mettler-Toledo or similar instrument. PXRD patterns were recorded with a PANalytical X Pert Pro X-ray diffractometer using Cu Kα radiation in reflection geometry (Bragg-Brentano). The sample is placed in a silicon single crystal sample holder of 0.2 mm depth and gently and precisely flattened. The tube voltage is 45 kV and current is 40 mA. The PXRD data are collected at room temperature in the range from 2θ=3.0°-40.0° with increments of 0.017° and measurement time of 20 s/step. PXRD data were analyzed using HighScore Plus 4.9 (Malvern Panalytical B.V.). Peak picking was performed with following parameters: minimum significance: 7.00, minimum tip width: 0.01° 2θ, maximum tip width: 1.00° 2θ, peak base width: 2.00° 2θ, top of smoothed peak.

### b) Quantification of Impurities

GC analysis was used for quantification of 2-propanol, xylene (mix of isomers), biphenyl, isopropyl palmitate. Instrument Agilent 6850-2 GC. Column: Agilent HP-5, 30 m×320 μm×0.25 μm. Oven temperature: 3 min at 70° C., 50 K/min→300° C.; 5 min at 300° C. Sample preparation: Samples were completely dissolved in 1,4-dioxane at 80° C. (filtration of turbid solution after cooling to 20° C.). Detector: FID. Calibration with external standards of xylene (mix of isomers), and biphenyl. HPLC analysis was used for quantification of TBPT and BBCT. Instrument: Agilent 1200 HPLC. Column: Phenomenex C18/5 μm, length 150 mm. Oven temperature: 40° C. Injection volume: 5 μL. Eluent: 900 parts acetonitrile+50 parts tetrahydrofuran+50 parts water (isocratic elution). Sample preparation: Samples were completely dissolved in tetrahydrofuran. Detector: DAD. Calibration with external standards of TBPT and BBCT.

### c) UV Absorption

The UV spectra have been recorded of micronized TBPT forms A and B as described in WO 2004/085412 A1 (Example 7: Preparation of a micronized UV absorber)

Thereby, d(50) is the mean particle size. In the case of UV-absorbers for cosmetics the spectral range between 290 nm and 400 nm is of most interest. The wavelength of the extinction maximum may be searched and the value of the extinction at this wavelength is taken for the characterization of the absorbing power of the substance. With the Beer-Lambert law, one obtains the molar decadic extinction coefficient at any wavelength with:

$$ε ( λ ) = \frac{E ( λ )}{c \cdot d} [ l / ( mol \cdot cm ] . \qquad ( I )$$

The respective specific extinction E1,1(λ) can be obtained via the following Equation:

$${E}_{1 , 1} ( λ ) = ε ( λ ) [ l / ( mol \cdot cm ) ] \cdot \frac{10 [ g / l ]}{M [ g / mol ]} \cdot l [ cm ] , \qquad ( II )$$

where M=molecular weight of the UV-absorber.

### Reference Example RE1—Preparation of 1,3,5-triazine, 2,4,6-tris[1,1′-biphenyl]-4-yl (TBPT)

Suitable materials for reactors/vessels are e.g., Hastelloy C or enamel steel or glass. 207 kg cyanuric chloride, 2080 kg biphenyl (molten) and 340 kg heptane are charged in a vessel (T=105-110° C.). 21 kg gaseous hydrochloric acid is dosed under the surface of the stirred mixture (T=105-110° C.). At 110° C., 300 kg aluminum chloride is added in portions depending on the temperature development. After finalizing the aluminum chloride dosing for ca. 4 h the reaction mass is stirred for approx. one hour at 110-114° C. until the reaction is completed. Afterward, heptane is removed under vacuum (>100 mbar) by distillation. The reaction mixture is hydrolyzed by carefully adding to a mixture of 2500 kg water, 135 kg sodium hydroxide and 1670 kg xylene at 85° C. The mixture is stirred at 85-90° C. for approx. 30 min until complete hydrolysis. The lower aqueous phase is separated from the organic product suspension at 90° C. Then the organic phase (suspension) is washed with 1000 kg water at 90° C. After separation of the aqueous phase, the organic phase (suspension) is dried by azeotropic distillation under vacuum. Water is completely removed from the suspension. By heating the yellow suspension to 143-150° C., the product dissolves and a turbid solution is obtained. Hot filtration is performed to remove traces of insoluble impurities. Within 4 h, the orange to brown solution is cooled to 40° C. to obtain the crystalline TBPT. The product is isolated by filtration, washed with 2000 kg xylene and dried at 100-140° C. under vacuum.

### Comparative Examples CE1-3: Usual Purification of TBPT

Purification of TBPT raw was performed in a Buchi Glass Oven Kugelrohr (BUCHI Labortechnik AG, 9230 Flawil, Switzerland).

CE1: 1-5 g TBPT raw synthesized according to the synthesis procedure of RE1 is heated under reduced pressure (<100 mbar, 1-22 h) until it becomes liquid (melting point 16 pprox. 285° C.). Volatile impurities are removed, and the product solidifies until cooling down to room temperature. The product turns dark the longer it is heated.

CE2: 1-5 g TBPT raw synthesized according to the synthesis procedure of RE1 is heated under reduced pressure (<100 mbar, 8-24 h) until 245-255° C. without melting. Volatile impurities are removed.

CE3: 1-5 g TBPT raw synthesized according to synthesis procedure of Example 1 is heated under reduced pressure (<10 mbar, 22 h) until 240° C. without melting. Volatile impurities are removed.

### Inventive Example: Purification of TBPT

Experimental set-up: 8 Liter stainless steel (Hastelloy) kneader reactor with counter hooks, filling volume 70% (approx. 1.9 kg TBPT), rotation speed 20/min. The kneader is heated-up with an oil thermostat. Off-gas is drawn out of the kneader and the gas stream is condensed in a spiral cooler. The product is always cooled down before opening of equipment to prevent contact to oxygen at higher temperature which could influence the color.

### Inventive Example IE1 (Solvent Methyl Ethyl Ketone)

### IE1_A: Transformation Polymorph A to Polymorph B

TBPT raw is loaded to the kneader reactor. The kneader reactor is evacuated and purged with nitrogen for inertization. There is a constant nitrogen purge of 10 L/h during the process. The kneader is heated-up with heating medium for product temperature above 243° C. for t_{T} hours at P_{T}. The temperature is measured in the product with several PT-sensors along the kneader to observe temperature distribution in the product and to control temperature above 243° C. as a minimum temperature of the complete product. Vacuum is held constant at 100-600 mbar.

The temperature (T_{T}) should not exceed 255° C. With higher temperatures the product color becomes dark, and the product gets nearer to the melting point at 280° C., which must be avoided. If the temperature gets to near to the melting point the product could become sticky. The machine is additionally insulated, so that no cold zones can occur. A good mixing of the material for constant temperature distribution and the removal of impurities is required.

TABLE 1
Preparation conditions for polymorph
B conversion of example IE1_A

| | Average | | |
|---|---|---|---|
| | product | | p_{T} average |
| | temperature T_{T} | t_{T} | pressure |
| | (° C.) | (h) | (mbar) |
| IE1_A_a | 246 | 4 | 383 |
| IE1_A_b | 244 | 2 | 563 |
| IE1_A_c | 250 | 4 | <600 |
| IE1_A_d | 245 | 2 | <600 |

The products from IE1_A_a, IE1_A_b, IE1_A_c, and IE1_A_d are removed from the reactor, mixed and homogenized to yield IE1_A_x.

### IE1_B: Transformation Polymorph B to Polymorph A

Part of the TBPT mixture IE1_A_x which was obtained from the example IE1_A is loaded to the kneader reactor. The kneader reactor is evacuated and purged with nitrogen for inertization. The pressure is adjusted to p₁ and the temperature is adjusted to T₁. Solvent is dosed with 10 to 30 mass percentage of the total inserted mass of TBPT to the kneader reactor. The solid product is wetted to a slurry with the solvent inside the apparatus. The temperature is kept below the boiling point of the solvent at P_{T}. The solvent should stay in the product mixture. The duration of mixing (t₁ at T₁) and the kind of solvent is variated process parameter for the different experiments. Afterwards the solvent is dried off at 140° C. and 100 mbar vacuum (1-6 h). The product is cooled down before opening of equipment to prevent contact to oxygen at higher temperature which could influence the color. Purified TBPT is analyzed.

TABLE 2
Preparation conditions for polymorph
A conversion of example IE1_B

| | | | Average | | |
|---|---|---|---|---|---|
| | | | product | | |
| | | Solvent/TBPT | temperature T₁ | t₁ | p₁ |
| | Solvent | (w %) | (° C.) | (h) | (mbar) |
| IE1_B_a | Methyl ethyl | 30:70 | 74 | 3.1 | 980 |
| | ketone | | | | |
| IE1_B_b | Methyl ethyl | 30:70 | 70 | 3.1 | 980 |
| | ketone | | | | |

### Inventive Example IE2 (Solvent Acetone)

### IE2_A: Transformation Polymorph A to Polymorph B

This process step is carried out as described in example IE1_A. The kneader reactor is opened to get a product sample of TBPT for analysis. The product remains in the reactor.

TABLE 3
Preparation conditions for polymorph
B conversion of example IE2_A

| | Average | | |
|---|---|---|---|
| | product | | p_{T} average |
| | temperature T_{T} | t_{T} | pressure |
| | (° C.) | (h) | (mbar) |
| IE2_A | 245 | 4 | <600 |

### IE2_B: Transformation Polymorph B to Polymorph A

This process step is carried out as described in example IE1_B with the exception that acetone was used as solvent.

TABLE 4
Preparation conditions for polymorph
A conversion of example IE2_B

| | | | Average | | |
|---|---|---|---|---|---|
| | | | product | | |
| | | Solvent/TBPT | temperature T₁ | t₁ | p₁ |
| | Solvent | (w %) | (° C.) | (h) | (mbar) |
| IE2_B | Acetone | 30:70 | 52 | 7.1 | 980 |

### Inventive Example IE3 (Solvent Ethanol)

### IE3_A: Transformation Polymorph A to Polymorph B

This process step is carried out as described in example IE1_A. The kneader reactor is opened to get a product sample of TBPT for analysis. The product remains in the reactor.

TABLE 5
Preparation conditions for polymorph
B conversion of example IE3_A

| | Average | | |
|---|---|---|---|
| | product | | p_{T} average |
| | temperature T_{T} | t_{T} | pressure |
| | (° C.) | (h) | (mbar) |
| IE3_A | 245 | 4 | <600 |

### IE3_B: Transformation Polymorph B to Polymorph A

This process step is carried out as described in example IE1_B with the exception that ethanol was used as the solvent.

TABLE 6
Preparation conditions for polymorph
A conversion of example IE3_B

| | | | Average | | |
|---|---|---|---|---|---|
| | | | product | | |
| | | Solvent/TBPT | temperature T₁ | t₁ | p₁ |
| | Solvent | (w %) | (° C.) | (h) | (mbar) |
| IE3_B | Ethanol | 30:70 | 72 | 4.1 | 980 |

### Inventive Example IE4 (Solvent Water)

### IE4_A: Transformation Polymorph A to Polymorph B

This process step is carried out as described in example IE1_A. The kneader reactor is opened to get a product sample of TBPT for analysis. The product remains in the reactor.

TABLE 7
Preparation conditions for polymorph
B conversion of example IE4_A

| | Average | | |
|---|---|---|---|
| | product | | p_{T} average |
| | temperature T_{T} | t_{T} | pressure |
| | (° C.) | (h) | (mbar) |
| IE4_A | 245 | 4 | <600 |

### IE4_B: Transformation Polymorph B to Polymorph A

This process step is carried out as described in example IE1_A with the exception that water was used as the solvent.

TABLE 8
Preparation conditions for polymorph
A conversion of example IE4_B

| | | | Average | | |
|---|---|---|---|---|---|
| | | | product | | |
| | | Solvent/TBPT | temperature T₁ | t₁ | p₁ |
| | Solvent | (w %) | (° C.) | (h) | (mbar) |
| IE4_B | Water | 30:70 | 140 | 9.1 | 2600 |

### Quantification of Impurities

TABLE 9
Overview of the purity of the respective examples in % and
content of impurities for 2,4-bis([1,1′-biphenyl]-
4-yl)-6-chloro-1,3,5-triazine (BBCT), biphenyl and xylene in ppm.

| | TBPT | BBCT | biphenyl | xylene |
|---|---|---|---|---|
| | (%) | (ppm) | (ppm) | (ppm) |
| RE1 | 98.4 | 1000 | 5000 | 5000 |
| CE1 | 99.5 | 200 | <50 | 400 |
| CE2 | | | | |
| CE3 | 99.5 | 300 | <50 | <100 |
| IE1_A_a | | | <200 | <200 |
| IE1_A_b | | | <200 | <200 |
| IE1_A_c | | | <200 | <200 |
| IE1_A_d | | | <200 | <200 |
| IE1_A_x | 98.5 | 500 | 100 | <100 |
| IE1_B_a* | 98.6 | 500 | 200 | <100 |
| IE1_B_b* | 97.8 | 600 | 300 | <100 |
| IE2_A | 98.9 | 500 | <100 | <100 |
| IE2_B** | 98.5 | 500 | 100 | <100 |
| IE3_A | 99.3 | 400 | <100 | <100 |
| IE3_B*** | 99.0 | 400 | <100 | <100 |
| IE4_A | 99.3 | 500 | <100 | <100 |
| IE4_B | 98.6 | 500 | <100 | <100 |

*contains 0.6-1% methyl ethyl ketone
**contains 0.7-1% acetone
***contains 0.3-1% ethanol

The concentration of impurities such as xylene, toluene, 1,1′-biphenyl, BBCT, is reduced after purification.

### Results

### DSC Measurement

TABLE 10
Comparison ΔH for DSC endothermic peaks at at
221-256° C. and at 282-293° C.

| | DSC endothermic | DSC endothermic |
|---|---|---|
| | peak at 221- | peak at 282- |
| | 256° C., | 293° C., |
| | ΔH [J/g] | ΔH [J/g] |
| RE1 | 43 | 59 |
| CE1 | 2 | 57 |
| CE2 | 5 | 58 |
| CE3 | 9 | 61 |
| IE1_A_a | | |
| IE1_A_b | 4 | 59 |
| IE1_A_c | 5 | 59 |
| IE1_A_d | 7 | 58 |
| IE1_A_x | 4 | 60 |
| IE1_B_a | 39 | 58 |
| IE1_B_b | 40 | 59 |
| IE2_A | 9 | 60 |
| IE2_B | 38 | 60 |
| IE3_A | 5 | 60 |
| IE3_B | 33 | 60 |
| IE4_A | 4 | 59 |
| IE4_B | 35 | 59 |

### PXRD Measurement

TABLE 11
Polymorph type determined on the basis of DSC and PXRD data

| | Polymorph type |
|---|---|
| | (DSC/PXRD) |
| RE1 | A |
| CE1 | B* |
| CE2 | B* |
| CE3 | B* |
| IE1_A_a | B |
| IE1_A_b | B |
| IE1_A_c | B |
| IE1_A_d | B* |
| IE1_A_x | B |
| IE1_B_a | A |
| IE1_B_b | A |
| IE2_A | B |
| IE2_B | A** |
| IE3_A | B |
| IE3_B | A** |
| IE4_A | B* |
| IE4_B | A** |

*Can contain residues of polymorph A. Complete transformation can be achieved by prolonged heat treatment as described in the transformation process step from Form A to Form B
**Can contain residues of polymorph B. Complete transformation can be achieved by prolonged treatment as described in the transformation process step from Form B to Form A

In CE1-3, polymorph A is transformed in polymorph B due to the high temperature. Only polymorph type A is suitable for sunscreen application due to the specific UV absorption properties (cf. FIG. 7). Moreover, polymorph type B is metastable at room temperature.

### XRD Peaks

TABLE 4
PXRD peaks of RE1

| 2θ [°], Cu Kα radiation | d spacing [Å] |
|---|---|
| 6.7 ± 0.2 | 13.1764 |
| 11.6 ± 0.2 | 7.61183 |
| 13.4 ± 0.2 | 6.58994 |
| 16.2 ± 0.2 | 5.4655 |
| 17.8 ± 0.2 | 4.97998 |
| 18.8 ± 0.2 | 4.71346 |
| 21.1 ± 0.2 | 4.20514 |
| 23.4 ± 0.2 | 3.80249 |
| 24.4 ± 0.2 | 3.65315 |
| 25.1 ± 0.2 | 3.5433 |
| 25.5 ± 0.2 | 3.4883 |
| 27.3 ± 0.2 | 3.26682 |
| 28.6 ± 0.2 | 3.12094 |
| 29.0 ± 0.2 | 3.08246 |
| 30.2 ± 0.2 | 2.95898 |
| 30.6 ± 0.2 | 2.92585 |
| 31.1 ± 0.2 | 2.87507 |
| 32.1 ± 0.2 | 2.79154 |
| 34.6 ± 0.2 | 2.5921 |
| 35.4 ± 0.2 | 2.53387 |
| 36.0 ± 0.2 | 2.49632 |
| 38.1 ± 0.2 | 2.36457 |
| 39.8 ± 0.2 | 2.26468 |

TABLE 5
PXRD peaks of CE3

| 2θ [°], Cu Kα radiation | d spacing [Å] |
|---|---|
| 6.7 ± 0.2 | 13.14058 |
| 8.6 ± 0.2 | 10.33751 |
| 10.9 ± 0.2 | 8.12938 |
| 13.2 ± 0.2 | 6.68518 |
| 13.5 ± 0.2 | 6.56906 |
| 16.0 ± 0.2 | 5.5431 |
| 17.5 ± 0.2 | 5.07011 |
| 18.5 ± 0.2 | 4.80271 |
| 18.9 ± 0.2 | 4.68455 |
| 19.5 ± 0.2 | 4.54677 |
| 19.9 ± 0.2 | 4.45175 |
| 20.7 ± 0.2 | 4.28169 |
| 21.7 ± 0.2 | 4.10418 |
| 22.1 ± 0.2 | 4.02839 |
| 23.1 ± 0.2 | 3.84939 |
| 23.9 ± 0.2 | 3.71963 |
| 24.1 ± 0.2 | 3.69373 |
| 25.4 ± 0.2 | 3.50319 |
| 26.1 ± 0.2 | 3.41193 |
| 26.7 ± 0.2 | 3.33773 |
| 27.2 ± 0.2 | 3.27654 |
| 27.8 ± 0.2 | 3.20755 |
| 28.7 ± 0.2 | 3.10639 |
| 30.5 ± 0.2 | 2.9317 |
| 32.5 ± 0.2 | 2.75526 |
| 35.2 ± 0.2 | 2.5464 |

TABLE 6
PXRD peaks of CE2

| 2θ [°], Cu Kα radiation | d spacing [Å] |
|---|---|
| 6.7 ± 0.2 | 13.16072 |
| 8.6 ± 0.2 | 10.34035 |
| 10.9 ± 0.2 | 8.1245 |
| 11.6 ± 0.2 | 7.63212 |
| 13.2 ± 0.2 | 6.68841 |
| 13.5 ± 0.2 | 6.57582 |
| 16.0 ± 0.2 | 5.54549 |
| 17.5 ± 0.2 | 5.07423 |
| 18.5 ± 0.2 | 4.80238 |
| 18.9 ± 0.2 | 4.68465 |
| 19.5 ± 0.2 | 4.54981 |
| 19.9 ± 0.2 | 4.45334 |
| 20.7 ± 0.2 | 4.28349 |
| 21.6 ± 0.2 | 4.10781 |
| 22.1 ± 0.2 | 4.02818 |
| 23.1 ± 0.2 | 3.85187 |
| 23.9 ± 0.2 | 3.72395 |
| 24.1 ± 0.2 | 3.69382 |
| 25.4 ± 0.2 | 3.50459 |
| 26.1 ± 0.2 | 3.41188 |
| 26.7 ± 0.2 | 3.33684 |
| 27.2 ± 0.2 | 3.27847 |
| 27.8 ± 0.2 | 3.20711 |
| 28.7 ± 0.2 | 3.10897 |
| 30.5 ± 0.2 | 2.93172 |
| 31.8 ± 0.2 | 2.81072 |
| 32.5 ± 0.2 | 2.7562 |
| 33.0 ± 0.2 | 2.71654 |
| 35.2 ± 0.2 | 2.54782 |
| 36.0 ± 0.2 | 2.49639 |
| 38.8 ± 0.2 | 2.32342 |

TABLE 7
PXRD peaks of TBPT pure batch IE1

| 2θ [°], Cu Kα radiation | d spacing [Å] |
|---|---|
| 6.7 ± 0.2 | 13.1397 |
| 11.6 ± 0.2 | 7.60021 |
| 13.4 ± 0.2 | 6.58451 |
| 16.2 ± 0.2 | 5.45921 |
| 17.8 ± 0.2 | 4.97608 |
| 18.8 ± 0.2 | 4.70904 |
| 21.1 ± 0.2 | 4.20194 |
| 23.4 ± 0.2 | 3.80046 |
| 24.4 ± 0.2 | 3.65157 |
| 25.1 ± 0.2 | 3.54119 |
| 25.6 ± 0.2 | 3.4845 |
| 27.3 ± 0.2 | 3.26341 |
| 28.6 ± 0.2 | 3.1193 |
| 29.0 ± 0.2 | 3.0799 |
| 29.8 ± 0.2 | 2.99888 |
| 30.2 ± 0.2 | 2.95746 |
| 30.6 ± 0.2 | 2.92438 |
| 31.1 ± 0.2 | 2.87592 |
| 32.1 ± 0.2 | 2.78857 |
| 32.8 ± 0.2 | 2.72906 |
| 34.6 ± 0.2 | 2.59104 |
| 35.4 ± 0.2 | 2.53382 |
| 36.0 ± 0.2 | 2.49532 |
| 38.1 ± 0.2 | 2.36412 |

1.-15. (canceled)

16. A composition comprising
1,3,5-triazine, 2,4,6-tris[1,1′-biphenyl]-4-yl (TBPT), and
a first impurity consisting of one or more halogen-comprising triazines, and
a second impurity consisting of biphenyl,
wherein the amount of the first impurity is equal to or less than 900 ppm by weight with respect to the total weight of the composition, and
wherein the amount of the second impurity is equal to or less than 4000 ppm by weight with respect to the total weight of the composition.

17. The composition according to claim 16, wherein
(1) the amount of the first impurity in the composition is equal to or less than 800 ppm by weight with respect to the total weight of the composition; or
(2) the halogen-comprising triazine is selected from chlorine-comprising triazines, bromine-comprising triazines, and a mixture thereof.

18. The composition according to claim 16, wherein the amount of the second impurity in the composition is equal to or less than 3000 ppm by weight with respect to the total weight of the composition.

19. The composition according to claim 16, wherein the composition further comprises a third impurity.

20. The composition of claim 19, wherein the third impurity comprises an aromatic hydrocarbon, wherein the third impurity does not comprise biphenyl, and wherein the amount of the third impurity in the composition is equal to or lower than 4000 ppm by weight with respect to the total weight of the composition.

21. The composition according to claim 21, wherein the aromatic hydrocarbon is selected from the group consisting of monocyclic-aromatic hydrocarbons, polycyclic aromatic hydrocarbons (PAH), and mixtures thereof, preferably is selected from the group consisting of alkyl-substituted benzene compounds (Cn-benzenes), condensed aromatic hydrocarbons, and mixtures thereof.

22. The composition according to claim 16, wherein at least a part of the TBPT is comprised in the composition in a crystalline form A, which, in an X-ray powder diffractogram at room temperature using Cu-Kα radiation, shows at least 3 of the 5 following reflexes, given in 20 values: 11.6±0.2, 17.8±0.2, 21.1±0.2, 23.4±0.2 and 24.4±0.2°θ.

23. The composition according to claim 16, wherein at least a part of the TBPT is comprised in the composition in a crystalline form B, which, in an X-ray powder diffractogram at room temperature using Cu-Kα radiation, shows at least 3 of the 5 following reflexes, given in 2θ values: 8.6±0.2, 10.9±0.2, 17.5±0.2, 18.5±0.2, 19.5±0.2, 19.9±0.2, and 20.7±0.2°θ.

24. The composition according to claim 16, wherein the composition further comprises a further compound, wherein the further compound is a solvent (S1) and has a boiling point B₁ at a pressure p_{B1} of 1 bar at a temperature T_{B1} equal to or lower than 235° C.

25. The composition according to claim 24, wherein the further compound has a melting point M₂ at a pressure p_{B2} of 1 bar at a temperature T_{B2} equal to or lower than 20° C.

26. The composition according to claim 24, wherein the further compound is selected from the list consisting of acetone, methyl ethyl ketone, ethanol, water, and mixtures thereof.

27. A process for interconverting crystalline form B of 2,4,6-tris(biphenyl-4-yl)-1,3,5-triazine (TBPT) into crystalline form A of TBPT comprising the steps of:
a) providing a composition C_{B} comprising TBPT in crystalline form B;
b) mixing the composition C_{B} in a solvent (S1) to obtain a first mixture;
c) stirring the first mixture at a temperature T₁ for a time t₁ and thereby obtaining TBPT in crystalline form A, and thereby providing a second mixture; and
d) isolating a composition C_{A} comprising TBPT in crystalline form A from the second mixture.

28. The process according to claim 27, wherein the TBPT providing step comprises
a1) providing in a second providing step a non-purified composition comprising TBPT and at least a first impurity, the first impurity consisting of one or more halogen-comprising triazine; and
a2) heating in a heating step the non-purified composition to a transition temperature TT range of from 235° C. to 280° C.

29. The process according to claim 28, wherein the heating step comprises keeping the pressure p₁ in the range of from 5 mbar to standard pressure.

30. A cosmetic product comprising the composition according to claim 16, wherein more than 80 wt-% of the TBPT is present in crystalline form A, which, in an X-ray powder diffractogram at room temperature using Cu-Kα radiation, shows at least 3 of the 5 following reflexes, given in 2θ values: 11.6±0.2, 17.8±0.2, 21.1±0.2, 23.4±0.2 and 24.4±0.2°θ, in a micronized form.

31. A UV filter in a cosmetic product for protecting hair or skin of a subject from the damaging effects of UV radiation comprising the composition of claim 16, wherein more than 80 wt-% of the TBPT is present in crystalline form A, which, in an X-ray powder diffractogram at room temperature using Cu-Kα radiation, shows at least 3 of the 5 following reflexes, given in 2θ values: 11.6±0.2, 17.8±0.2, 21.1±0.2, 23.4±0.2 and 24.4±0.2°θ.
